(19) United States
(12) Patent Application Publication
(10) Pub. No.: US 2024/0299594 A1
(43) Pub. Date: Sep. 12, 2024

(54) PROCESS FOR THE MANUFACTURING OF A GADOLINIUM COMPLEX SOLUTION

(71) Applicant: BRACCO IMAGING SPA, Milan (IT)

(72) Inventors: Andrea BANIN, Collegno (IT); Andrea BARALE, Bibiana (IT); Valeria BOI, Strambino (IT); Sonia GAZZETTO, Cascinette d'Ivrea (IT); Federica BUONSANTI, Torino (IT)

(73) Assignee: BRACCO IMAGING S.P.A., Milan (IT)

(21) Appl. No.: 18/566,057

(22) PCT Filed: Jul. 26, 2022
(86) PCT No.: PCT/EP2022/070902

§ 371 Date: Nov. 30, 2023

(30) Foreign Application Priority Data
Jul. 27, 2021 (EP) 21187887.1

(51) Int. Cl. A61K 49/10; A61K 9/08; C07F 5/00

(52) U.S. Cl. CPC A61K 49/106; A61K 9/08; C07F 5/003

(57) ABSTRACT

The invention relates to a process for the manufacturing of a solution of a dimeric C gadolinium complex, such as [μ-[1-[bis[2-(hydroxy-xO)-3-[4,7,10-tris[(carboxy-xO)methyl]-1,4,7,10-tetraazacyclododec-1-yl-κN¹,κN⁴, κN⁷,κN¹⁰]propyl]amino]-1-deoxy-D-glucitolate(6-)]]digadoliniumcomplex, which is useful in the field of diagnostic imaging and of contrast agents in Magnetic Resonance Imaging (MRI), comprising the steps of precipitating a portion of free gadolinium metal ions by means of a precipitating agent. The invention further relates to a process for isolating the dimeric gadolinium complex from said solution, and to the solution and the isolated dimeric gadolinium complex obtainable by such processes.

### TECHNICAL FIELD

The present invention relates to a process for the manufacturing of a solution of a dimeric gadolinium complex, such as [p-[1-[bis[2-(hydroxy-κO)-3-[4,7,10-tris[(carboxy-κO)methyl]-1,4,7,10-tetraazacyclododec-1-yl-κN¹,κN⁴,κN⁷,κN¹⁰]propyl]amino]-1-deoxy-D-glucitolate(6-)]]digadolinium complex, characterized by great robustness and suitable for large scale production. The present invention further relates to a process for isolating the dimeric gadolinium complex from said solution. The dimeric gadolinium complex is useful in the field of diagnostic imaging and of contrast agents in Magnetic Resonance Imaging (MRI).

### Backqround of the invention

Magnetic Resonance Imaging (MRI) is a well-known diagnostic imaging technique increasingly used in clinical diagnostics for a growing number of indications.

Gadolinium (Gd(III)) complexes are commonly used as contrast agents in MRI due to their long relaxation times. However, the gadolinium metal ion [Gd(H₂O)₈]3'⁰ is extremely toxic for living organism even at low doses (10-20 micromol/kg).

Thus, in order to be considered as a potentially valuable MRI contrast agent, a Gd(III) complex shall display a high thermodynamic (and possibly kinetic) stability in to prevent the release of the toxic metal ion. Moreover, processes for manufacturing the Gd(III) complex are advantageous when they allow effective and efficient removal of the toxic metal ion that is present within the reaction mixture after the complexation step.

WO 2017/098044 discloses dimeric paramagnetic complexes useful as contrast agents in MRI. These dimeric complexes, in particular the dimeric Gd(III) complexes, show increased relaxivity compared to non specific contrast agents currently in use in the daily diagnostic practice. Accordingly, such dimeric Gd(III) complexes could be potentially used for in vivo diagnostic imaging at doses lower than those required by the contrast agents currently in use.

WO 2017/098044 further discloses a preparation process for the dimeric paramagnetic complexes therein disclosed. Such process comprises the step of complexing the ligand in water with stoichiometric addition of a suitable Gd(III) derivative, such as a Gd(III) salt or oxide. The solution comprising the complex is then filtered and evaporated under reduced pressure. The crude product is then purified on an adsorbent resin, such as Amberchrome CG161M, and the fractions containing the product are finally pooled and evaporated.

The solution obtained after the complexation step and purification step of the process disclosed in WO 2017/098044 may contain a substantial amount of mono-gadolinated complexes.

Mono-gadolinated complexes are Gd(III) complexes wherein the dimeric ligand disclosed in WO 2017/098044 chelates only one gadolinium ion instead of two. These mono-gadolinated complexes do not show the favourable relaxometric properties of the di-gadolinated Gd(III) complexes disclosed in WO 2017/098044. Thus, it would be advantageous to remove (or substantially reducing the amount of) these mono-gadolinated complexes from the solution obtained from the complexation step. However, methods for removal of mono-gadolinated complexes from the reaction mixture are not completely satisfactory, in that complete removal of mono-gadolinated complexes can hardly be achieved (this is i.a. due to the fact that mono-gadolinated complexes have very similar physical characteristics to di-gadolinated complexes). For this reason, it would be advantageous to provide a method for the manufacture of the di-gadolinated Gd(III) complexes disclosed in WO 2017/098044 which substantially avoids the generation of mono-gadolinated complexes during or after the complexation step as much as possible.

Moreover, the reproducibility of complexation step disclosed in WO 2017/098044 depends on the precise weighing of the reactants of the complexation step and on the precise determination of the titles thereof. At least for this reason, the robustness of the process disclosed in WO 2017/098044 could be improved.

Accordingly, there is the need of a process for the preparation of the Gd(III) complexes disclosed in WO 2017/098044 that overcomes the above mentioned problems, in particular a process that is reproducible, robust, so that it is particularly advantageous for large-scale production of the dimeric paramagnetic complexes disclosed in WO 2017/098044, while possibly limiting as much as possible generation of mono-gadolinated complexes.

### SUMMARY OF THE INVENTION

In a first aspect, the present invention provides a process for the manufacturing of a solution of the gadolinium complex of formula I

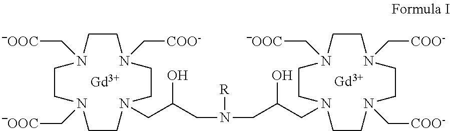

wherein R is a C₃-C₁₂ hydroxyalkyl comprising at least 2 hydroxyl groups, preferably R is a C₅-C₇ polyol;
comprising the following steps:
i) providing a solution of a dimeric ligand of formula Ia

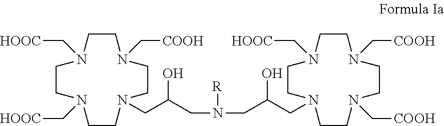

wherein R is as defined above,
ii) adding to the solution of the previous step a molar excess of gadolinium metal ions to complex the dimeric ligand provided in step i), whereby an intermediate solution comprising the gadolinium complex of formula I is obtained, and
iii) adding to the intermediate solution of the previous step a precipitating agent to precipitate a portion of free gadolinium metal ions as gadolinium salt, whereby said solution of gadolinium complex of formula I is obtained,
   wherein the precipitating agent is selected from the group consisting of phosphate (PO4³⁻), monohydrogen phosphate (HPO₄²⁻), dihydrogen phosphate (H₂PO₄⁻), orthophosphoric acid (H₃PO₄), oxalate (C₂O₄²⁻), hydrogen oxalate (HC₂O₄⁻) and oxalic acid (H₂C₂O₄).

In a preferred aspect, the present invention provides a process for the manufacturing of a solution of the following gadolinium complex (Compound 1)

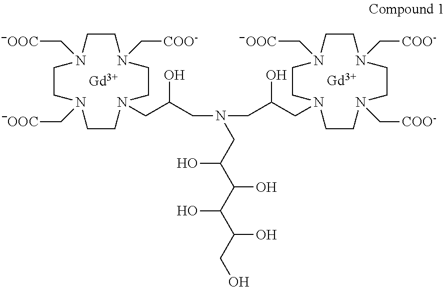

comprising the following steps:
i) providing a solution of the following dimeric ligand (Compound 1a)

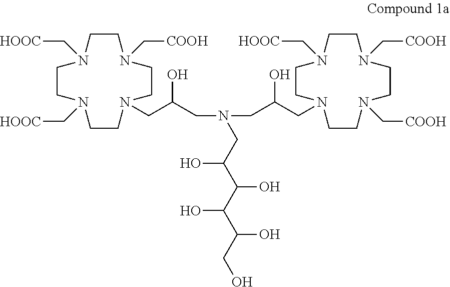 [SMILES: O=C(O)CN1CCN(CC(=O)O)CCN(CC(O)CN(CC(O)CN2CCN(CC(=O)O)CCN(CC(=O)O)CCN(CC(=O)O)CC2)CC(O)C(O)C(O)C(O)CO)CCN(CC(=O)O)CC1]

ii) adding to the solution of the previous step a molar excess of gadolinium metal ions to complex the dimeric ligand provided in step i), whereby an intermediate solution comprising the gadolinium complex of formula I is obtained, and
iii) adding to the intermediate solution of the previous step a precipitating agent to precipitate a portion of free gadolinium metal ions as gadolinium salt, whereby said solution of gadolinium complex of formula I is obtained, wherein the precipitating agent is selected from the group consisting of phosphate (PO₄³⁻), monohydrogen phosphate (HPO₄²⁻), dihydrogen phosphate (H₂PO4⁻), orthophosphoric acid (H₃PO₄), oxalate (C₂O₄²⁻), hydrogen oxalate (HC₂O₄⁻), and oxalic acid (H₂C₂O₄).

In a second aspect, the present invention provides a solution of gadolinium complex of formula I

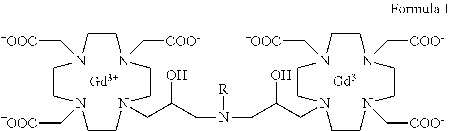

wherein R is as defined above,
obtainable according to the process for manufacturing a solution of gadolinium complex according to any of its embodiments.

In a third aspect, the present invention provides a process for the manufacturing of an isolated gadolinium complex of formula I

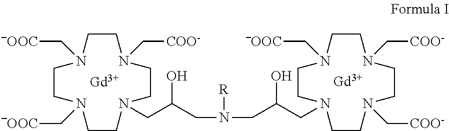

wherein R is as defined above,
comprising the process of manufacturing of a solution of the gadolinium complex as herein disclosed in any of its embodiment, and the further subsequent step iv):
iv) isolating the gadolinium complex from the purified solution obtained in said step iii).

In a further aspect, the present invention provides an isolated gadolinium complex of formula I

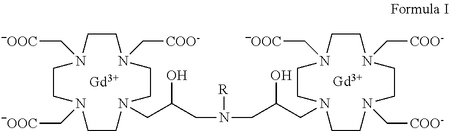

wherein R is as defined above,
obtainable according to the process for the manufacturing of an isolated gadolinium complex according to any of its embodiment.

These and further aspects, along with embodiments thereof, are disclosed in more details in the following section.

### DETAILED DESCRIPTION OF THE INVENTION

As used herein, and unless otherwise provided, the term “mono-gadolinated complex” refers to a complex having the same structure as the dimeric complex of formula I, or Compound 1, but chelating only one gadolinium metal ion instead of two. For example, a mono-gadolinated complex is a compound of the general formula Ic

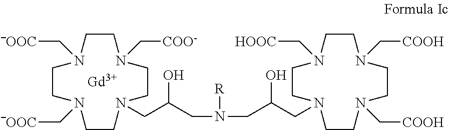

wherein R is as defined above for Formula I.

As used herein, and unless otherwise provided, the term “precipitating agent” refers to the agent added in step iii) that is, or that generates, an anion at least in the conditions of step iii) when added to the intermediate solution according to the process of the invention. Such anion is able to generate, through ionic bond(s) with the free gadolinium metal ions, a gadolinium salt as herein defined. The precipitating agent is selected from the group consisting of phosphate (PO₄³⁻), monohydrogen phosphate (HPO₄²⁻), dihydrogen phosphate (H₂PO₄⁻), orthophosphoric acid (H₃PO₄), oxalate (C₂O₄²⁻), hydrogen oxalate (HC₂O₄₋), and oxalic acid (H₂C₂O₄).

As used herein, and unless otherwise provided, the term “gadolinium salt” refers to the salt generated after addition of the precipitating agent as herein defined. The gadolinium salt comprises as a cation Gd³⁺, and as a counter-anion the anion which is, or is generated by, the precipitating agent. At least in the conditions of step iii) of the process of the invention, and preferably also in the conditions of the steps downstream of step iii), the gadolinium salt is present within the reaction mixture in a solid and filterable physical form. Examples of gadolinium salt are gadolinium phosphate and gadolinium oxalate.

As used herein, and unless otherwise provided, the term “free gadolinium metal ions” refers to gadolinium ions, such as [Gd(H₂O)₈]³⁺, that are present within a solution and that are not chelated by the dimeric ligands.

As used herein, and unless otherwise provided, the term “intermediate solution” refers to the solution comprising the gadolinium complex of formula I obtained after the complexation step (step ii)) but before the purification step (step iii)).

In the present description, and unless otherwise provided, the expression “alkyl” comprises within its meaning any linear or branched hydrocarbon chain. For example, “C₁-C₆ alkyl” comprises within its meaning a linear or branched chain comprising from 1 to 6 carbon atoms such as: methyl, ethyl, propyl, iso-propyl, butyl, iso-butyl, tert-butyl, pentyl, iso-pentyl, tert-pentyl, n-hexyl, and the like. In the present description, and unless otherwise provided, the term “tBu” refers to the C₄ alkyl tert-butyl (or 1,1-dimethylethyl).

The term “hydroxyalkyl” (or “polyol”, as used herein interchangeably) comprises within its meaning any of the corresponding linear or branched hydrocarbon chain wherein one or more hydrogen atoms are replaced by hydroxyl groups.

For instance, and unless otherwise provided, the expression “C₃-C₁₂ polyol” (or “C₃-C₁₂ polyhydroxyalkyl”) comprises within its meaning any of the corresponding C₃-C₁₂ linear or branched hydrocarbon chain in which 2 or more, e.g. from 2 to 11 hydrogen atoms have been replaced by hydroxyl groups. Among them, C₃-C₁₀ polyols are preferred, and C₅-C₇ polyols are particularly preferred. Examples of C₅-C₇ polyols include pentyl-polyols (or polyhydroxypentyls) such as pentyl-diols, pentyl-triols, pentyl-tetraols and pentyl-pentaol, respectively comprising from 2, 3, 4 and 5 hydroxyl groups on a Cₛ alkyl chain; hexyl-polyols (or polyhydroxyhexyls) analogously comprising from 2 to 6 hydroxyl groups on a C₆ alkyl chain; and heptyl-polyols (or polyhydroxyheptyls) comprising from 2 to 7 hydroxyl groups on a C₇ alkyl chain.

In the present description the term “protecting group” designates a protective group adapted for preserving the function of the group to which it is bound. Specifically, protective groups can be used to preserve amino, hydroxyl or carboxyl functions.

Appropriate carboxyl protective groups may thus include, for example, benzyl, alkyl e.g. tert-butyl or benzyl esters, or other substituents commonly used for the protection of such functions, which are all well known to those skilled in the art [for a general reference, T.

W. Green and P. G. M. Wuts; Protective Groups in Organic Synthesis, Wiley, N.Y. 1999, third edition].

Moreover, the terms “moiety” or “moieties”, “residue” or “residues” are herewith intended to define the residual portion of a given molecule once properly attached or conjugated, either directly or through any suitable linker, to the rest of the molecule.

The compounds herein disclosed, e.g. the compounds of formula I and Compound 1, may have one or more asymmetric carbon atom, otherwise referred to as a chiral carbon atom, and may thus give rise to diastereomers and optical isomers. Unless otherwise provided, the present invention further includes all such possible diastereomers as well as their racemic mixtures, their substantially pure resolved enantiomers, all possible geometric isomers, and pharmaceutical acceptable salts thereof.

The present invention further relates to a process for the manufacture of a solution of complex of the formula I, or Compound 1, in which each of the acidic groups contained therein, e.g. on R, may be deprotonated. In such case, the acidic groups contained in the dimeric ligand of formula Ia, or Compound 1a, may be in the respective form, e.g. deprotonated.

The present invention further relates to a process for the manufacture of a solution of complex of the formula I, or Compound 1, in which each of the basic groups contained therein, e.g. the tertiary amine, may be protonated. In such case, the basic groups contained in the dimeric ligand of formula Ia, or Compound 1a, may be in the respective form, e.g. protonated.

The present invention refers to a process for the manufacturing of a solution of the gadolinium complex of formula I

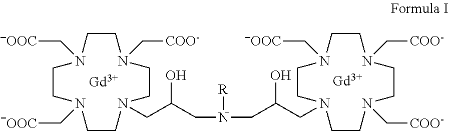

wherein R is a C₃-C₁₂ hydroxyalkyl comprising at least 2 hydroxyl groups; comprising the steps of i) providing a solution of a dimeric ligand of formula Ia

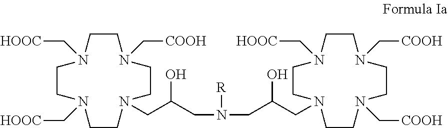

wherein R is as defined above; ii) adding to the solution of the previous step a molar excess of gadolinium metal ions to complex the dimeric ligand provided in step i), whereby an intermediate solution comprising the gadolinium complex of formula I is obtained, and iii) adding to the solution of the previous step at least a precipitating agent to precipitate a portion of free gadolinium metal ions as gadolinium salt, thereby obtaining the solution of the gadolinium complex of formula I, wherein the precipitating agent is selected from the group consisting of phosphate (PO4³⁻), monohydrogen phosphate (HPO₄²⁻), dihydrogen phosphate (H₂PO4-), orthophosphoric acid (H₃PO₄), oxalate (C₂O₄²⁻), hydrogen oxalate (HC₂O₄₋), and oxalic acid (H₂C₂O₄).

The process of the invention is robust, thus overcoming the problems of the prior art process. Indeed, the burden of the precise weighing of the reactants of the complexation step and of the determination of titles is heavily reduced due to the addition of a molar excess of gadolinium metal ions. As the process of the invention is robust, reproducible and efficient, it can be more easily implemented for large-scale production. Moreover, by adding a molar excess of gadolinium metal ions, the presence of mono-gadolinated complex within the final product is notably lowered compared to addition in stoichiometric amounts.

Adding a molar excess of gadolinium metal ions provides a higher amount of free gadolinium metal ions after complexation compared to the addition of a stoichiometric quantity, or less of a stoichiometric quantity, of gadolinium metal ions; however, it has been found that this higher amount of free gadolinium metal ions can be effectively and efficiently removed by carrying out step iii) of the invention, i.e. by precipitating free gadolinium metal ions with a precipitating agent as herein disclosed. Accordingly, combining step ii) and step iii) provides a particularly effective process of the invention, in that a solution comprising the complex of formula I with low amounts of both mono-gadolinated complex and of free gadolinium metal ions is obtained via a process that is robust and suitable for large-scale production.

Applicant has also found that by precipitating free gadolinium metal ions with methods not according to the invention for the manufacturing of a solution of the complex of formula I and for removing free gadolinium ions, a solution containing a high and thus unsuitable amount of free gadolinium metal ions and/or of mono-gadolinated complex is obtained. In particular, as it is also demonstrated in the experimental section below by means of comparative examples, methods known in the prior art to precipitate gadolinium ions might not be suitable to obtain a solution of the gadolinium complex of formula I comprising suitable amounts of free gadolinium metal ions and/or of mono-gadolinated complex. For example, it is known e.g. by “Preparation, Purification, and Characterization of Lanthanide Complexes for Use as Contrast Agents for Magnetic Resonance Imaging”, Averill et al., Journal of Visualized Experiments, that free gadolinium metal ions precipitate as Gd(OH)₃ when the pH of a solution comprising free gadolinium metal ions is raised to a sufficiently high pH. However, as it is demonstrated in the experimental section below by means of comparative examples, this method of precipitation of free gadolinium metal ions by basification is not effective when it is used on a solution of the gadolinium complex of formula I, as gadolinium hydroxide do not precipitate. Accordingly, this basification method does not reduce the amount of free gadolinium metal ions within the solution of the gadolinium complex of interest, i.e. of formula I.

Applicant has surprisingly found that by adding a precipitating agent for precipitating a portion of free gadolinium metal ions as a gadolinium salt, wherein the precipitating agent is selected from the group consisting of phosphate (PO₄³⁻), monohydrogen phosphate (HPO₄²⁻), dihydrogen phosphate (H₂PO₄⁻), orthophosphoric acid (H₃PO₄), oxalate (C₂O₄²⁻), hydrogen oxalate (HC₂O₄₋), and oxalic acid (H₂C₂O₄), preferably in the amounts disclosed below, it is possible to effectively remove high amounts of free gadolinium metal ions without generating a high amount of mono-gadolinated complex, in particular when the pH is adjusted and/or maintained in the ranges disclosed below during and/or after the precipitation step.

The process of the invention allows precipitating a portion of free gadolinium metal ions, and in particular a substantial portion thereof. Indeed, as showed in the Experimental section below, the precipitation step iii) allows precipitating a substantial portion of free gadolinium metal ions that is present after the complexation step ii), whereby the content of free gadolinium metal ions is reduced from almost tens of thousands ppm to just above a hundred ppm or even tens ppm (vs. the amount of gadolinium complex). Accordingly, the process of the invention provides for manufacturing a solution comprising the gadolinium complex as herein disclosed containing low amounts of free gadolinium metal ions, and in particular the amount of free gadolinium metal ions within such solution after the precipitation step iii), and before the optional further purification step(s) after step iii), can be less than 350 ppm, preferably less than 150 ppm, more preferably less than 100 ppm, and even more preferably less than 80 ppm, vs. the amount of gadolinium complex. According to the present invention, high ppm values of free gadolinium metal ions, e.g. ppm values of 4000 ppm or higher, may be determined by conventional complexometric titration with EDTA in the presence of xylenol orange, while lower ppm values of free gadolinium metal ions, e.g. ppm values lower than 4000 ppm, are preferably determined by carrying out the HPLC Procedure 1 as set out in the Experimental section below.

Moreover, the process of the invention provides for manufacturing a solution comprising the gadolinium complex as herein disclosed, wherein the amount of mono-gadolinated complex within such solution is low, namely lower than 550 ppm, preferably lower than the limit of quantitation (LoQ) of the analytical method used to quantify the mono-gadolinated complex, i.e. less than 400 ppm vs. the amount of gadolinium complex. These ppm values of mono-gadolinated complex, as well as all ppm values of mono-gadolinated complex in the present invention, are determined by carrying out the HPLC Procedure 2 as set out in the Experimental section below. Such a low amount of mono-gadolinated complexes within the final solution of the process of the invention, i.e. less than 550 ppm, preferably less than 400 ppm of mono-gadolinated complexes vs. the amount of gadolinium complex, has no significant or even no negative impact on the relaxivity of the final, isolated complex.

Advantageously, step iii) of the process of the invention provides for manufacturing a solution of gadolinium complex as herein disclosed, wherein within such solution, the amount of free gadolinium metal ions is less than 150 ppm, preferably less than 125 ppm, more preferably less than 80, and even more preferably less than 50 ppm, vs. the amount of gadolinium complex, and the amount of mono-gadolinated complex is less than 400 ppm vs. the amount of gadolinium complex.

Preferred compounds of formula I and Ia include compounds in which R is a C₃-C₁₂ polyhydroxyalkyl (or C₃-C₁₂ polyol) having from 2 to 11 and, preferably, from 3 to 10 hydroxyl groups on the C₃-C₁₂ alkyl chain. Preferably, R is the residue of a C₅-C₇ polyol e.g. selected from pentyl-polyols (or polyhydroxypentyls) comprising at least 2, and preferably from 2 to 4 hydroxyl groups on the Cₛ alkyl chain; hexyl-polyols comprising at least 2, and preferably from 2 to 5 hydroxyl groups on the C₆ alkyl chain; or heptyl-polyols comprising at least 2 and, and preferably from 3 to 6 hydroxyl groups on the C₇ alkyl chain.

In one preferred embodiment, the process of the invention is for manufacturing a solution of the gadolinium complex of formula I wherein R of formulae I and Ia is a C₅-C₇ polyol, preferably selected from a pentyl-tetraol of formula

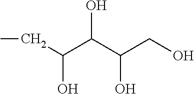

and a hexyl-pentaol of formula

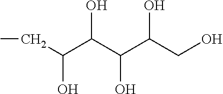

comprising the steps i), ii) and iii) as herein disclosed according to any embodiment thereof.

In a particularly preferred embodiment, the process of the invention is for manufacturing a solution of the following gadolinium complex (Compound 1)

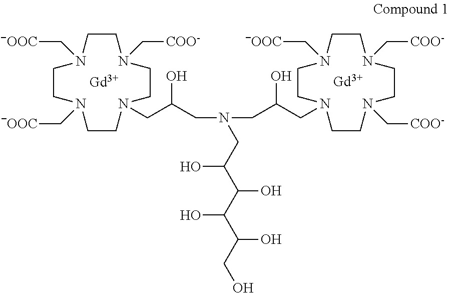

comprising steps i), ii) and iii) as herein disclosed according to any embodiment thereof. Compound 1 showed great relaxivity as demonstrated in WO 2017/098044, and is thus particularly preferred.

When the process of the invention is a process for the manufacturing of a solution of the gadolinium complex Compound 1, the dimeric ligand provided in step i) is the correspondent dimeric ligand Compound 1a

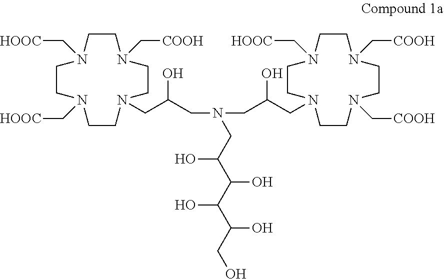 [SMILES: O=C(O)CN1CCN(CC(=O)O)CCN(CC(O)CN(CC(O)CN2CCN(CC(=O)O)CCN(CC(=O)O)CCN(CC(=O)O)CC2)CC(O)C(O)C(O)C(O)CO)CCN(CC(=O)O)CC1]

Step i) of the process of the invention, i.e. providing a solution of a dimeric ligand of formula Ia, or of Compound 1a, can be performed for example by carrying out a known process for preparing a solution of non-complexed dimeric ligand of formula Ia, or of Compound 1a such as disclosed in WO 2017/098044. Preferably, the dimeric ligand is provided by deprotecting the correspondent protected dimeric ligand according to the method for deprotecting as herein disclosed.

The solution provided in step i), as well as the intermediate solution and the final solution of the gadolinium complex as manufactured in the process of the invention, are preferably aqueous solutions.

Step ii) of the process of the invention is performed by adding to the solution of the previous step a molar excess of gadolinium metal ions to complex the dimeric ligand provided in step i), thus obtaining an intermediate solution comprising the gadolinium complex of formula I, or Compound 1. Accordingly, step ii) of the process of the invention provides for the complexation of the dimeric ligand of formula Ia, or of any other dimeric ligand as herein disclosed, with gadolinium.

Since the dimeric ligand provided in step i) has two chelating moieties, and one dimeric ligand can thus chelate two gadolinium metal ions, the term “molar excess” when referring to step ii) of the process of the invention refers to an amount of moles of gadolinium metal ions that is more than twice than the amount of moles of the dimeric ligand. Accordingly, the term “molar excess”, when referring to step ii) of the process of the invention, refers to more than 2 moles of gadolinium metal ions with respect to 1 mole of dimeric ligand. For example, 2.05 moles or more, preferably from 2.05 to 2.50 moles, more preferably up to 2.20 moles, and even more preferably up to 2.12 moles of gadolinium metal ions are added to the solution with respect to 1 mole of dimeric ligand provided in step i).

According to step ii) of the process of the invention, gadolinium metal ions can be added for example by adding a gadolinium derivative, such as a soluble gadolinium salt, to the solution. Suitable gadolinium derivatives can be, for example, an oxide such as Gd₂03, or a soluble gadolinium salt such as GdC1₃.

Step ii) is preferably carried out maintaining the temperature of the solution within the range from 20 to 50° C., more preferably from 30 to 45° C., and even more preferably from 37 to 43° C. After adding the gadolinium metal ions according to step ii), the reaction mixture is preferably maintained, for example at the temperature ranges provided above, for a time from 1 to 5 hours, more preferably from 2 to 4 hours, before carrying out the subsequent step(s).

During and/or after the addition of gadolinium metal ions of step ii), the pH is preferably adjusted to and/or maintained in the range from 5.0 to 7.0, more preferably from 5.0 to 6.0, for example for a time and/or at the temperature as provided above. This pH adjustment and/or maintenance can be done for example by adding a suitable base, e.g. sodium hydroxide, to the solution of step ii).

According to a preferred embodiment, after step ii), and optionally before step iii), the process of the invention comprises the further step of desalting the (intermediate) solution of the gadolinium complex, preferably via nanofiltration. This desalting (e.g. nanofiltration) step allows removing the salts produced in complexation step, e.g. the salts generated after addition of the soluble gadolinium salt, as well as the salts generated in the optional deprotection method (if carried out). The desalting step does not remove free gadolinium metal ions nor mono-gadolinated complexes, and is useful to remove salts in order to improve the subsequent optional steps of treating the solution to remove the precipitating agent.

The desalting step can be carried out until the value of conductivity of the solution is 5.0 mS/cm or lower, preferably 1 mS/cm or lower, and even more preferably 0.8 mS/cm or lower.

Step iii) of the process of the invention provides for iii) adding to the intermediate solution of the previous step a precipitating agent to precipitate a portion of the free gadolinium metal ions. Indeed, a portion of free gadolinium metal ions precipitates as gadolinium salt, so that a solution of gadolinium complex of formula I is obtained with low amounts of free gadolinium metal complex, for example the amount in ppm provided above. In order to obtain a precipitation of free gadolinium metal ions and to avoid generation of mono-gadolinated complexes, the precipitating agent has to be at least one selected from the group consisting of phosphate (PO₄³⁻), monohydrogen phosphate (HPO₄²⁻), dihydrogen phosphate (H₂PO₄⁻), orthophosphoric acid (H₃PO₄), oxalate (C₂O₄²⁻), hydrogen oxalate (HC₂O₄₋), and oxalic acid (H₂C₂O₄). Preferably, the precipitating agent is at least one anion selected from the group consisting of phosphate (PO₄³⁻), oxalate (C₂O₄²⁻), and monohydrogen phosphate (HPO₄²⁻), and more preferably is monohydrogen phosphate (HPO₄²⁻).

The precipitation step (step iii)) provides for removing from the solution a portion of the free gadolinium metal ions in excess that have not reacted in the previous complexation step (step ii)), by means of precipitation of the free gadolinium metal ions.

The precipitating agent can be added for example by mixing a solution comprising the precipitating agent with the intermediate solution, or by directly adding the precipitating agent, for example when the latter is comprised in a precipitating salt, to the intermediate solution.

As used herein, and unless otherwise provided, the term “precipitating salt” refers to a salt added in step iii) that comprises the precipitating agent as an anion and any suitable counter-cation. The precipitating salt is soluble at least in the intermediate solution and within the conditions of step iii), so that it is able to solubilize and release the precipitating agent within the intermediate solution. For example, a preferred precipitating salt is Na₂HPO₄, which comprises the precipitating agent monohydrogen phosphate (HPO₄²⁻) as an anion, and sodium as a counter-cation.

If the precipitating agent is added in step iii) by addition of a precipitating salt comprising the precipitating agent, suitable counter-cations of the precipitating salt include for example cations selected from alkali metals, alkaline-earth metals, ammonium, and organic cations. For example, counter-cations of the precipitating salt can be selected from sodium and potassium; sodium being particularly preferred. The precipitating salt can be preferably selected from the group consisting of sodium phosphate (Na₃PO₄), potassium phosphate (K₃PO₄), sodium hydrogen phosphate (Na₂HPO₄), potassium hydrogen phosphate (K₂HPO₄), sodium dihydrogen phosphate (NaH₂PO₄), potassium dihydrogen phosphate (KH₂PO₄), sodium oxalate (Na₂C₂O₄), potassium oxalate (K₂C₂O₄), sodium hydrogen oxalate (NaHC₂O4), and potassium hydrogen oxalate (KHC₂O₄₋).

According to step iii) of the process of the invention, the precipitating agent is preferably added at least in stoichiometric amounts with respect to the free gadolinium metal ions within the intermediate solution. Advantageously, the precipitating agent is added in an amount of at least of 1.1 moles, preferably in an amount from 1.1 to 5 moles, more preferably from 1.2 to 3 moles, even more preferably from 1.4 to 2.5 moles, and most preferably from 1.4 to 1.6 moles, with respect to 1 mole of gadolinium metal ions within the intermediate solution. As demonstrated in the experimental section also by means of comparative examples, adding these preferred amounts of precipitating agent provides solutions containing both low amounts of free gadolinium metal ions, i.e. amounts lower than the ones specified above, and of mono-gadolinated complexes, i.e. amounts lower than 550 ppm, preferably lower than the LoQ of the method used to determine the amount of mono-gadolinated complexes (<400 ppm vs. the amount of gadolinium complex), after the optional filtration step and before the optional further purification steps. On the contrary, when the precipitating agent is added in step iii) in greater amounts with respect to the preferred amounts above, the resulting solution might contain a high amount of mono-gadolinated complex, i.e. an amount higher than 600 ppm.

When step iii) of the process of the invention is carried out by adding the preferred amounts of precipitating agent as specified above, the process of the invention may preferably comprise the further step of determining the amount of free gadolinium metal ions within the intermediate solution before adding the precipitating agent, whereby the precipitating agent can be added in the preferred amount as specified above. This determination step can be carried out according to known methods for determining the amount of free gadolinium metal ions, for example according to the method herein disclosed.

Step iii) is preferably carried out by maintaining the temperature of the solution within the range from 15 to 40° C., more preferably from 20 to 30° C. After adding the precipitating agent according to step iii), the reaction mixture is preferably maintained, for example at the temperature ranges provided above, for a time from 1 to 4 hours, preferably from 1.5 to 3 hours, more preferably of 2 hours, before carrying out the optional subsequent step(s).

In a preferred embodiment, during and/or after the addition of the precipitating agent according to step iii), the pH is adjusted to and/or maintained at a value of 4.5 or higher, preferably of 4.7 or higher, more preferably of 4.9 or higher, even more preferably of 5.5 or higher, for example for a time and/or a temperature as provided in paragraph above. Preferably, this pH is maintained at least until the precipitated gadolinium salt is filtered out from the solution of the gadolinium complex. As demonstrated in the experimental section below also by means of comparative examples, applicant has surprisingly found that precipitating free gadolinium metal ions while adjusting and/or maintaining the pH at these values, a solution containing low amounts of mono-gadolinated complexes after the optional filtration step and before the optional further purification steps is obtained, for example a solution containing an amount lower than 550 ppm, preferably and lower than 400 ppm of mono-gadolinated complexes vs. the gadolinium complex.

According to a further preferred embodiment, during and/or after the addition of the precipitating agent according to step iii), the pH can be adjusted to and/or maintained such that it is higher than the values indicated above, and that is 10.0 or lower, preferably 9.0 or lower, more preferably 8.5 or lower, even more preferably 7.5 or lower, and most preferably 6.5 or lower, for example for a time and/or a temperature as provided above. Preferably, this pH is maintained at least until the precipitated gadolinium salt is filtered out from the solution of the gadolinium complex. Applicant has surprisingly found that by operating below these pH values, the amount of free gadolinium metal ions within the solution after the precipitation step and before the optional further purification steps is lowered.

According to a more preferred embodiment, during and/or after the addition of the precipitating agent according to step iii), the pH can be adjusted to and/or maintained in the range from 4.5 to 9.0, more preferably from 4.7 to 8.5, even more preferably from 4.9 to 7.3, and most preferably from 6 to 6.5 or from 5.5 to 6.5, for example for a time and/or a temperature as provided above. Preferably, this pH is maintained at least until the precipitated gadolinium salt is filtered out from the solution of the gadolinium complex. Applicant has surprisingly found that adjusting and/or maintaining the pH within the ranges indicated above allows obtaining solutions having a particular low content of free gadolinium metal ions and of mono-gadolinated complexes after the optional filtration step and before the optional further purification steps, for example a content lower compared to the same process wherein the pH is not adjusted and/or maintained at such pH.

The pH adjustment can be done for example by adding a suitable acid, e.g. HCl, or a suitable base, e.g. NaOH, to the solution. This adjustment is particularly useful to counteract the possible pH changes caused by the addition of the precipitating agent. It is evident that if the addition of the precipitating agent does not cause a change in pH such that the pH of the resulting solution falls outside the preferred ranges disclosed above (e.g. because the pKa of the precipitating agent is within the preferred values above and/or because the precipitating agent is added in low amounts whereby the pH of the resulting solution does not fall outside the preferred ranges disclosed above), then pH adjustment may be not necessary.

Preferably, the pH according to the preferred values disclosed above is maintained at least until the precipitated gadolinium salt is filtered out from the solution of the gadolinium complex.

In a further preferred embodiment, after step iii), the process of the invention further comprises the step of filtering the obtained solution of gadolinium complex to remove the gadolinium salt from the solution, whereby the gadolinium salt is separated from such solution. This filtration step can be carried out according to any filtration method known in the art, for example by using pharmaceutical membrane filters.

In a further preferred embodiment, the process of the invention comprises the further step of treating the solution of gadolinium complex of formula I obtained after step iii) to remove, if present, the precipitating agent that have not reacted with the free gadolinium metal ions to form the gadolinium salt. This treatment step does not remove free gadolinium metal ions nor mono-gadolinated complexes.

This treatment step can be carried out for example by loading the solution of gadolinium complex on an ionic exchange resin, preferably at a flow rate from 1 to 3 BV/h.

Alternatively, or together with loading the complex on an ionic exchange resin, the treatment step can be carried out by (A) adding to the solution of gadolinium complex a precipitating cation, different from gadolinium metal ions Gd³⁺, to precipitate the anion that is, or is generated by, the precipitating agent, whereby at least part of such anion precipitates as a salt together with the precipitating cation, and (B) removing the salt thus formed by means of a filtration step, such as the one disclosed above. Advantageously, only one filtration step can be carried out to remove both the gadolinium salt and the salt formed by the precipitating agent and the precipitating cation.

As used herein, and unless otherwise provided, the term “precipitating cation” refers to a cation, different from gadolinium metal ions Gd³⁺, that is added during an optional step of treatment of the solution of gadolinium complex of formula I. The precipitating cation is able to generate, together with the anion that is, or is generated by, the precipitating agent, a salt that is in a solid physical form at least in the conditions of the reaction mixture during the addition of the precipitating cation.

The addition of the precipitating cation can be carried out for example by adding to the solution of gadolinium complex of formula I a soluble salt that contains the precipitating cation as the cation thereof, and/or by mixing a solution comprising the precipitating cation with the solution of gadolinium complex of formula I.

The precipitating cation can be suitably selected by the skilled person based on the salt formed by the ionic bond between the precipitating cation and the anion that is, or is generated by, the precipitating agent. Indeed, any suitable precipitating cation can be used, as long as the salt formed by the precipitating cation and such anion precipitates at least in the conditions of the reaction mixture during the addition of the precipitating cation, whereby at least part of the precipitated salt can be removed e.g. by filtration. For example, the precipitating cation Ca²⁺can be added to the solution of gadolinium complex according to the optional treating step above disclosed: when the pH of the solution reaches about 9, a salt formed by such anion and the precipitating cation Ca²⁺precipitates, so that it can be later removed e.g. by filtration. According to this example, the precipitating cation Ca²⁺can be added to the solution of gadolinium complex of formula I as a soluble salt, or preferably as hydroxide, e.g. as Ca(OH)₂, that solubilizes once it is added to the solution, thus releasing the precipitating cation Ca²⁺In an embodiment, the method for providing a solution of gadolinium complex of the invention comprises at least one further purification step after step iii), and preferably after the treatment step for removal of the precipitating agent (if carried out). This further purification step is useful to further reduce the residual amount of free gadolinium metal ions that is present after the precipitation of step iii), in order to provide a solution of gadolinium complex with a content of free gadolinium metal ions as low as possible. In particular, according to this embodiment of the method of the invention, the larger portion of free gadolinium metal ions is removed by the precipitation step iii) (wherein the amount of free gadolinium metal ions is reduced from thousands of ppm to few hundreds, or even to tens of ppm, vs. gadolinium complex), and a smaller portion thereof is removed by the at least one further purification step.

For example, this further purification step can comprise loading the solution of gadolinium complex onto a suitable resin, such as an adsorbent resin (for example Amberlite XAD1600), whereby a further portion of residual free gadolinium metal ions is removed from the solution. Preferably, before loading the solution onto the resin, the solution is concentrated (e.g. by distilling the aqueous solvent under vacuum) until the amount of gadolinium complex is within the range from 15 to 30% w/w, more preferably from 20 to 25% w/w.

In a further embodiment, after step iii), and optionally after the at least one further purification step, the method for providing a solution of gadolinium complex of the invention comprises the step of treating the solution of gadolinium complex with carbon. This step allows removal of endotoxins and promotes discoloration of the solution.

In another aspect, the present invention refers to a solution of gadolinium complex of formula I, wherein R is as above defined, or Compound 1, obtainable according to any embodiment of the process for manufacturing of the solution as herein disclosed. Preferably, within the solution of the invention obtainable according to any embodiment of the process of the invention as herein disclosed, the amount of free gadolinium metal ions is less than 350 ppm, preferably less than 150 ppm, more preferably less than 100 ppm, and even more preferably less than 80 ppm, vs. the amount of gadolinium complex, and/or the amount of mono-gadolinated complex is less than 550 ppm, preferably less than 400 ppm vs. the amount of gadolinium complex. A solution of gadolinium complex of formula I, wherein R is as above defined, or of Compound 1, comprising the amounts of free gadolinium metal ions and/or of mono-gadolinated complex as above provided, is also a further aspect of the present invention.

In a further aspect, the present invention refers to a process for the manufacturing of an isolated gadolinium complex of formula I

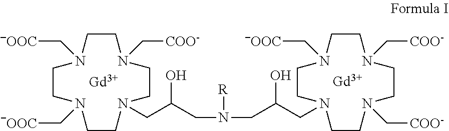

wherein R is as defined above, or Compound 1, comprising the process of manufacturing of a solution of the gadolinium complex as herein disclosed according to any embodiment thereof, and further step iv):
iv) isolating the gadolinium complex.

The process for the manufacturing of an isolated gadolinium complex of the invention allows obtaining the isolated gadolinium complexes herein disclosed by means of a robust and efficient process suitable for large-scale production.

The isolation step (step iv)) can be carried out by any suitable isolation method known to the skilled person that allows separating the complex from the solvent of the solution obtained in said step iii).

For example, the isolation step (step iv)) can be carried out by drying the solution of gadolinium complex of formula I (or Compound 1), possibly under vacuum, for example as disclosed in WO 2017/098044. The crude complex so obtained can then be further dried e.g. in an oven, whereby the gadolinium complex is obtained as a powdered solid.

In an additional aspect, the present invention refers to an isolated gadolinium complex of formula I

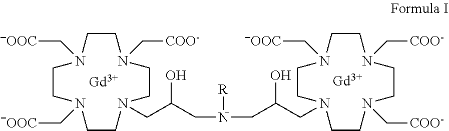

wherein R is as above defined, or Compound 1, obtainable according to any embodiment of the process for manufacturing of the isolated gadolinium complex as herein disclosed. In a further aspect, the invention refers to a method for deprotecting a protected dimeric ligand of formula Ib

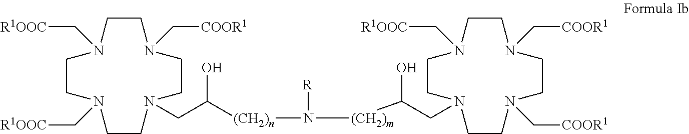

wherein R is a C₃-C₁₂ hydroxyalkyl comprising at least 2 hydroxyl groups, preferably R is a C₅-C₇ polyol, for example selected from a pentyl-tetraol of formula

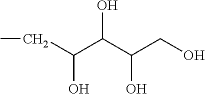

and a hexyl-pentaol of formula

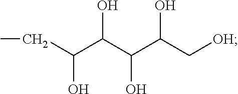

n is 1 or 2, preferably n is 1;
m is 1, 2, 3, 4, 5 or 6, preferably m is 1; and
R¹ is a C₁-C₆ alkyl, preferably a C₄ alkyl, and more preferably is t-butyl,
to obtain a solution of the correspondent dimeric Iigand of formula Id

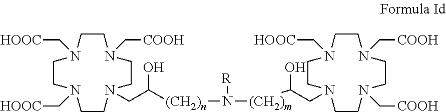

wherein R, n, and m are as defined above for Formula Ib,
comprising the following steps:
a) providing a solution, preferably an aqueous solution, of the protected dimeric ligand;
b) adding an acid, preferably an inorganic acid, to the solution of the previous step to lower the pH thereof;
c) during and/or after step b), heating and/or maintaining the temperature of the reaction mixture to a temperature higher than 40° C., preferably higher than 40° C. and up to 60° C., more preferably within the range from 45 to 55° C., to deprotect the protected dimeric ligand, whereby a solution comprising the dimeric ligand is obtained; and
d) optionally neutralizing the dimeric ligand, for example by adding a base, such as NaOH, to the solution comprising the dimeric ligand obtained in step c), whereby the carboxyl groups of the dimeric ligand are protonated.

According to a preferred embodiment of the method for deprotection, in formulae Ib and Id, n and m are independently 1 or 2, and more preferably both n and m are 1; in this latter more preferred case, formula Id corresponds to formula Ia reported above.

According to a further preferred embodiment of the deprotecting method, the protected dimeric ligand is Compound 1b

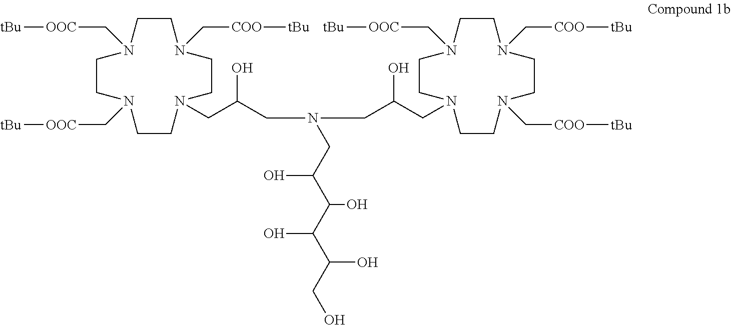

and the obtained solution is of the correspondent dimeric ligand is Compound 1a

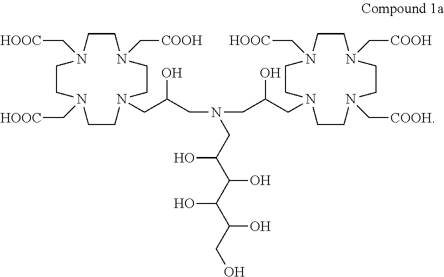 [SMILES: O=C(O)CN1CCN(CC(=O)O)CCN(CC(O)CN(CC(O)CN2CCN(CC(=O)O)CCN(CC(=O)O)CCN(CC(=O)O)CC2)CC(O)C(O)C(O)C(O)CO)CCN(CC(=O)O)CC1]

As showed in the Experimental Section, this deprotection method, and in particular step c), is very advantageous, because it allows obtaining very short deprotection times, in particular reaction times below 24 hours, for example within the range from 8 to 20 hours, preferably from 12 to 18 hours, more preferably 16 hours.

Moreover, this deprotection method, and in particular step c), provides for deprotecting the protected dimeric ligand by using low amounts of acid during step b). Indeed, an amount from 10 to 45 moles, preferably from 10 to 35 moles, more preferably from 15 to 25 moles of acid, such as the ones above, and preferably of HCl, vs. 1 mol of the protected dimeric ligand can be used in this deprotection method. This provides the advantage of saving reagents, as well as reducing the production of salts during the deprotection method. For example, when HCl is used in step b) as the acid, using a low amount of HCl will reduce the subsequent amount of NaCl salt that is formed when NaOH is used in optional step d) to neutralize the dimeric ligand; similar examples can also be brought when other acids are used in step b).

Preferably, the acid added in step c) is an inorganic acid, such as H₂SO₄, H₃PO₄, HCl, HBr and the likes. Inorganic acid comprising a counterion having a single negative charge, such as HCl, HBr and the likes, are particularly preferred, in particular when the method for deprotecting is used upstream of the method for manufacturing a solution of gadolinium complex as detailed herein, as they tend not to interact with free gadolinium metal ions, and as they can be more easily removed during the purification processes (for example by means of nanofiltration).

In view of the many advantages of this deprotecting method, the solution of the dimeric ligand of the method for manufacturing a solution of gadolinium complex as herein described is preferably provided by carrying out the deprotecting method as herein disclosed according to any of its embodiments, in particular when both m and n of formulae Ib and Id are 1, or when the protected dimeric ligand is Compound 1b.

According to an embodiment of the deprotecting method, when HCl is used as acid, HCl is added to the solution of step b) as a 34% w/w hydrochloric acid aqueous solution.

According to a preferred embodiment of the deprotecting method, the starting concentration of the protected dimeric ligand within the solution of step a) is comprised in the range from 5% to 20% (w/w), preferably in the range from 12% to 18% (w/w).

According to a preferred embodiment of the deprotecting method, optional step d) (neutralization of the dimeric ligand) can be carried out by adjusting the pH of the reaction mixture to a value comprised in the range from 4 to 7, preferably from 5 to 6, more preferably from 5.3 to 5.7, even more preferably to 5.5. This is preferably done by adding a suitable amount of a base, such as NaOH, to reach the pH mentioned above.

After step c) of the deprotecting method, i.e. after hydrolyzing the C₁-C₆ alkyl group R¹ (or the C₄ alkyl tBu, when Compound 1b is the protected dimeric ligand) a solution comprising the correspondent deprotected dimeric ligand and the correspondent alcohol of R^{l} (or of tBu) is obtained. Thus, according to a further preferred embodiment, after step c), and preferably after optional step d) (when carried out), the correspondent alcohol of R^{l}, such as tBuOH (when R¹ is tBu), is removed from the solution comprising the deprotected dimeric ligand, preferably by distilling such solution. According to a preferred embodiment, the solution comprising the dimeric ligand is distilled until the final concentration of dimeric ligand is comprised in the range from 8% to 12% (w/w), more preferably from 9% to 11% (w/w), and even more preferably is 10% (w/w).

### Experimental Section

The following examples will help to further illustrate the invention and are not meant to limit the scope thereof.

### Example 1—Deprotection of the Dimeric Ligand

To a mixture (1501.56 g) of the protected dimeric ligand Compound 1b (186.08 g, 0.141 mol)

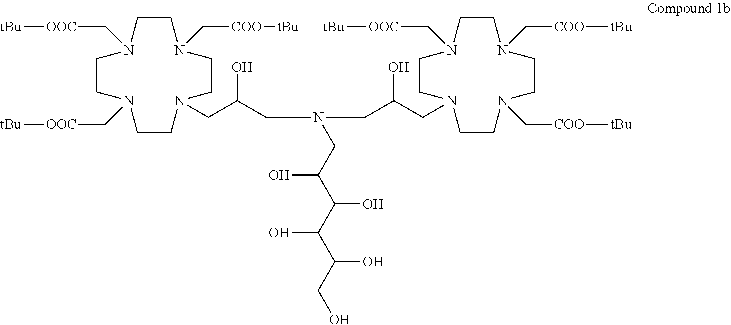

at a concentration of 1²0.4% (w/w) in water, 34% w/w hydrochloric acid aqueous solution (435.65 g, 4.06 mol, 30 eq. mol vs Compound 1b) is added maintaining the temperature at 30° C. At the end of the addition, the mixture is heated to 50° C. and kept under stirring for 16 h. After complete deprotection, 30% w/w sodium hydroxide aqueous solution is added until pH 5.6, and Compound 1a is obtained. The t-butanol formed as by-product is removed by distillation. The solution containing Compound 1a is concentrated by distillation at 50° C. under vacuum until the final concentration of about 10% (w/w).

### Example 2—Preparation of a Solution of the Dimeric Gadolinium Complex

The solution of the dimeric ligand 1-[bis[2-hydroxy-3-[4,7,10-tris(carboxymethyl)-1,4,7,10-tetraazacyclododec-1-yl]propyl]amino]-1-deoxy-D-glucitol (compound 1a)

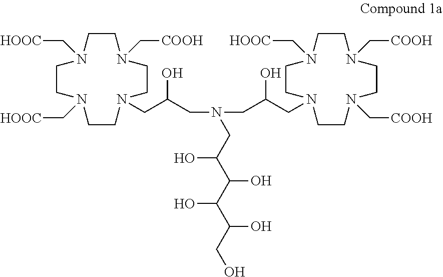 [SMILES: O=C(O)CN1CCN(CC(=O)O)CCN(CC(O)CN(CC(O)CN2CCN(CC(=O)O)CCN(CC(=O)O)CCN(CC(=O)O)CC2)CC(O)C(O)C(O)C(O)CO)CCN(CC(=O)O)CC1]

as obtained by Example 1 is loaded into a first reactor and heated to 40° C. Gadolinium chloride solution (2.1 mol vs. 1 mol of Compound 1a) is added maintaining the temperature in the range of 37-43° C. At the end of the addition, the pH is adjusted to 5.5 by adding 10% w/w sodium hydroxide aqueous solution. The mixture is maintained at 40° C. for 3 h.

An intermediate solution comprising the gadolinium complex [p-[1-[bis[2-(hydroxy-κO)-3-[4,7,10-tris[(carboxy-κO)methyl]-1,4,7,10-tetraazacyclododec-1-yl-κN¹,κN⁴,κN⁷,κN¹⁰]propyl]amino]-1-deoxy-D-glucitolate(6-)]]digadolinium (Compound 1) is thus obtained, and the amounts of mono-gadolinated complex (MonoGd) and free gadolinium metal ions (Free Gd) are measured.

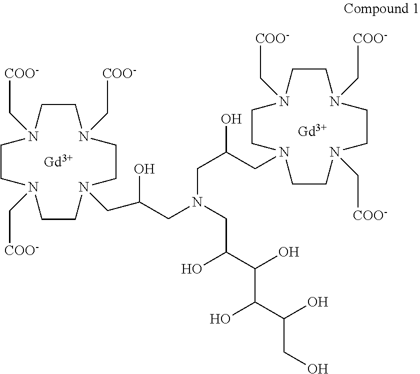

Then, the salts produced in Example 1 and in complexation steps are removed by nanofiltration; diafiltration is performed until the value of conductivity is lower than 1.0 mS/cm. This desalting step does not remove free gadolinium metal ions nor mono-gadolinated complexes. At the end of the nanofiltration, the mixture is concentrated until 10÷12% w/w and 1.5 mol/mol of Na₂HPO₄ vs. free gadolinium metal ions (free Gd) are added to the solution. After addition of Na₂HPO₄, the pH of the solution is measured for each trial and is reported in Table I below (column “pH start”); formation of a white precipitate is observed. The pH is then adjusted to the value reported in Table I in the column “pH fin”. The mixture is kept under stirring for 2 h. Finally, the suspension is filtered and the amounts of mono-gadolinated complex (MonoGd) and free gadolinium metal ions (Free Gd) are measured. These amounts, as well as the amounts obtained after complexation, are reported in Table I below.

TABLE I

| | | Free Gd | MonoGd | | |
|---|---|---|---|---|---|
| | | (ppm vs | (ppm vs | pH | PH |
| Trial | | Compound 1) | Compound 1) | start | fin |
| 1 | Starting | 19480 | n.q. | 7.89 | 6.13 |
| | After filtration | 32 | n.q. | | |
| 2 | Starting | 13150 | n.q. | 7.23 | 6.10 |
| | After filtration | 70 | n.q. | | |
| 3 | Starting | 17052 | n.q. | 6.78 | 6.08 |
| | After filtration | 52 | n.q. | | |
| 4 | Starting | 7721 | n.q. | 7.30 | 7.30 |
| | After filtration | 70 | n.q. | | |
| 5 | Starting | 10427 | n.q. | 7.20 | 4.90 |
| | After filtration | 10 | n.q. | | |
| 6 | Starting | 11888 | n.q. | 6.50 | 4.54 |
| | After filtration | 15 | 538 | | |
| 7 | Starting | 18037 | n.q. | 8.30 | 8.30 |
| | After filtration | 250 | n.q. | | |
| 8 | Starting | 7628 | n.q. | 8.04 | 8.04 |
| | After filtration | 318 | n.q. | | |
| 9 | Starting | 6030 | n.q. | 6.50 | 6.50 |
| | After filtration | 75 | n.q. | | |
| 10 | Starting | 8136 | n.q. | 7.87 | 5.71 |
| | After filtration | 24 | n.q. | | |
| 11 | Starting | 9753 | n.q. | 7.17 | 5.73 |
| | After filtration | 60 | n.q. | | |

On basis of the results reported in Table I, it is possible to observe that there is a significant reduction of the amount of Free Gd for all trials i to 11 provided above. All the solutions of trials 1 to 5 and 7 to 11 contain a non-quantifiable amount of Mono-Gd, i.e. an amount of less than 400 ppm of Mono-Gd; trial 6 shows that at pH 4.54 there is an amount of MonoGd higher than the LoQ, i.e. an amount of 538 ppm vs Compound 1. Table I also shows that an amount of MonoGd <than the LoQ and low amounts of FreeGd are obtained maintaining the pH within the range of 4.9-8.3.

### Example 3—Removal of Residual Phosphates

The solutions of trials 1-9 obtained in Example 2 are loaded on ionic exchange resin (Diaion PA 308, previously activated) at the flow rate of 1+3 BV/h. Removal of most of the residual phosphates from the solutions is thus obtained.

### Example 4⁻Removal of Residual Phosphates

To the solutions of trials 1-9 obtained in Example 2, Ca(OH)₂ (1 mol/mol vs Na₂HPO₄) is added and pH increases until about 9. Formation of an insoluble white precipitate comprising the precipitating anions (and not gadolinium) is thus observed. The mixture is kept under stirring for 2 h. Then, the suspension is filtered and the cake is washed with water, thus achieving removal of most of the residual phosphates.

### Example 5—Further Purification and Isolation of Dimeric Gadolinium Complex

The solutions of Example 3 are loaded in a second reactor, the pH of each solution is adjusted to 5.7+6.3 by diluted HCl addition, and water is distilled at 45+55° C. under vacuum until the assay of the gadolinium complex is about 20+25% w/w. The concentrated solutions are loaded with a flow rate of 0.5 BV/h on Amberlite XAD1600 (amount of resin: 30 mL/g of product), previously activated. The purification is performed with water and mixture of isopropanol and water.

The fractions with high purity (evaluation on HPLC-FLD/UV) are loaded into another reactor. After preliminary concentration a treatment with carbon is performed. The suspensions are filtered in order to remove the carbon and the solutions are concentrated under vacuum at 45+55° C. until 25% w/w concentration.

The gadolinium complex Compound 1 is finally isolated by drying under vacuum the solutions containing it.

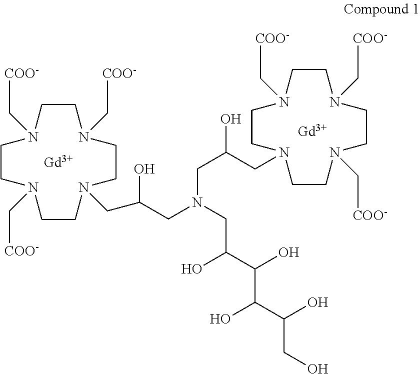

### Example 6—Precipitation of Free Gadolinium Ions with Disodium Oxalate (Comparative)

An example using excessive amount of precipitating agent, such as sodium oxalate, as comparative precipitation step is performed, this time adjusting the pH after addition of disodium oxalate.

To a 20% w/w solution of Compound 1 containing free gadolinium as obtained by complexation with the reaction conditions of Example 1, the amounts of mono-gadolinated complex (MonoGd) and free gadolinium metal ions (Free Gd) are measured and reported in Table IV below. Then, an excessive amount of disodium oxalate is added (10 mol vs 1 mol free Gd).

After oxalate addition, the pH rises from 5.91 until 8.45 and formation of a white solid is immediately observed. The pH is then reduced until about 6 with HCl 1N and maintained as such. At the end of the addition of sodium oxalate, the mixture is cooled down to 5° C. and maintained at this temperature for 2 h.

After the suspension is filtered, the content of mono-gadolinated complex and free gadolinium metal ions are measured and showed in Table IV.

TABLE IV

| | Free Gd | MonoGd |
|---|---|---|
| Trial | ppm vs Compound 1 | ppm vs Compound 1 |
| Starting | 10765 | n.d. |
| After filtration | 112 | 470 |

According to the results showed in Table IV above, the precipitation of free gadolinium metal ions with oxalate provides a solution comprising an amount of mono-gadolinated complex higher than the LoQ. As stated above, mono-gadolinated complexes do not show the favourable properties of the (di-gadolinated) Gd(III) complexes, e.g. of complex Compound 1. Accordingly, the precipitation of free gadolinium metal ions with an excessive amount of precipitating agent, such as an excessive amount of oxalate, provides a solution containing an amount of mono-gadolinated complexes that is over the LoQ, i.e. over 400 ppm.

### Example 7—Precipitation of Free Gadolinium Ions with Tripotassium Phosphate

To a 10% w/w solution of Compound 1 containing free gadolinium as obtained by complexation with the reaction conditions of Example 2, the content of mono-gadolinated complex (MonoGd) and free gadolinium metal ions (FreeGd) are measured. Then, K₃PO4 is added (1.5 mol vs mol of free Gd) to the solution.

After phosphate addition, the pH rises from 5.42 until 9.00 and formation of a white solid is immediately observed. The mixture is maintained at pH 9.00 under stirring for 2 h at rT and, then, the solid is filtered obtaining a solution. The content of free gadolinium metal ions (Free Gd) and mono-gadolinated complex (MonoGd) is measured and reported in Table V below.

TABLE V

| | Free Gd | MonoGd |
|---|---|---|
| Trial | ppm vs Compound 1 | ppm vs Compound 1 |
| Starting | 13744 | n.q. |
| After filtration | 136 | n.q. |

On basis of the results reported in Table V, it is possible to observe that, with the precipitation with phosphate as a precipitating agent, there is a significant reduction of the amount of Free Gd. Moreover, the solution contains a non-quantifiable amount of Mono-Gd, i.e. an amount of less than 400 ppm of Mono-Gd. Table V further shows that it is possible to obtain a low amount of FreeGd, and amounts of MonoGd <than the LoQ, at pH 9.0.

### Example 8—Precipitation of Free Gadolinium Ions with Potassium Hydrogen Tartrate (Comparative)

To a 10% w/w solution of Compound 1 containing free gadolinium as obtained by complexation with the reaction conditions of Example 2, potassium hydrogen tartrate is added (2 mol vs mol of free Gd) and a slight suspension is observed.

After hydrogen tartrate addition, the pH decreases from 5.42 until 4.21. The mixture is maintained under stirring for 2 h at rT and, then, the solid is filtered obtaining a solution. The content of free gadolinium metal ions (Free Gd) and mono-gadolinated complex (MonoGd) is measured and reported in Table VI below.

TABLE VI

| | Free Gd | MonoGd |
|---|---|---|
| Trial | ppm vs Compound 1 | ppm vs Compound 1 |
| Starting | 13744 | n.q. |
| After filtration | 10412 | n.q. |

According to the results showed in Table VI above, the precipitation of free gadolinium metal ions with hydrogen tartrate provides a solution containing a high amount of free gadolinium metal ions that is not satisfactory, because such high amount of free gadolinium metal ions cannot be efficiently and effectively reduced to suitable pharmaceutical amounts, e.g. by means of the at least one further purification step.

### Example 9—Precipitation of Free Gadolinium Ions with Disodium Hydrogen Citrate (Comparative)

To a 10% w/w solution of Compound 1 containing free gadolinium as obtained by complexation with the reaction conditions of Example 2, disodium hydrogen citrate is added (1.5 mol vs. mol of free Gd).

No precipitation is observed, and thus the trial is interrupted. As no precipitation is observed, the amount of free gadolinium metal ions within the solution of Compound 1 is deemed too high to be satisfactory, because such high amount of free gadolinium metal ions cannot be efficiently and effectively reduced to suitable pharmaceutical amounts, e.g. by means of the at least one further purification step.

### Example 10—Precipitation of Free Gadolinium Ions with Sodium Acetate (Comparative)

To a 10% w/w solution of Compound 1 containing free gadolinium as obtained by complexation with the reaction conditions of Example 2, sodium acetate is added (4.5 mol vs. mol of free Gd).

No precipitation is observed, and thus the trial is interrupted. As no precipitation is observed, the amount of free gadolinium metal ions within the solution of Compound 1 is deemed too high to be satisfactory, because such high amount of free gadolinium metal ions cannot be efficiently and effectively reduced to suitable pharmaceutical amounts, e.g. by means of the at least one further purification step.

### Example 11—Precipitation of Free Gadolinium Ions as Gd(OH)₃ by Basification (Comparative)

30% NaOH solution is added to a 16% w/w solution of Compound 1 containing 7000 ppm of free gadolinium vs. Compound 1 until pH 8.65.

No precipitation is observed.

The test above was repeated using several solutions containing (i) different concentrations of Compound 1 (in particular, concentrations of Compound 1 ranging from 16 to 20% w/w) and (ii) different amounts of free gadolinium metal ions (in particular, free gadolinium metal ions ranging from 230 to 25000 ppm vs. Compound 1).

In all cases, no precipitation is observed. Accordingly, although gadolinium ions are known to precipitate as Gd(OH)₃ in basic conditions, this precipitation by basification procedure cannot be used to suitably reduce free gadolinium metal ions on a solution of the gadolinium complex as herein defined, such as a solution of Compound 1.

### Example 12—Precipitation of Free Gadolinium Ions with Disodium Oxalate

To a 10% w/w solution of Compound 1 containing free gadolinium as obtained by complexation with the reaction conditions of Example 2, the content of mono-gadolinated complex (MonoGd) and free gadolinium metal ions (FreeGd) are measured. Then, disodium oxalate is added (2.25 mol/mol vs free Gd) to the solution.

After oxalate addition, the pH rises from 5.56 until 7.78 and formation of a white solid is immediately observed. The pH is then reduced until 6.43 with HCl 1N. At the end the mixture is maintained at room temperature for 2 h.

After the suspension is filtered, the content of mono-gadolinated complex and free gadolinium metal ions are measured. The content of mono-gadolinated complex (MonoGd) and of free gadolinium metal ions (Free Gd) are reported in Table VII below.

TABLE VII

| | Free Gd | MonoGd |
|---|---|---|
| Trial | ppm vs Compound 1 | ppm vs Compound 1 |
| Starting | 11730 | n.d. |
| After filtration | 64 | n.q. |

Table VII clearly shows that carrying out a trial as above, and in particular a trial involving oxalate as a precipitating agent in suitable amounts, provides a solution comprising very low amounts of Free Gd and an amount of MonoGd below the LoQ.

### HPLC Procedure 1—Determination of the Amount of Free Gadolinium Metal Ions (Free-Gd)

The determination of the amount of free gadolinium metal ions in relation to the amount of the gadolinium complex (e.g. of Compound 1) is performed by reverse phase HPLC (High Performance Liquid Chromatography) with FLD (Fluorescence Detector) detection. The use of EDTA (ethylenediaminetetraacetic acid) in the mobile phase ensures the formation of the Gd(EDTA) complex if free Gd(III) is present in the sample.

Chromatographic Conditions

| Instrument | Agilent 1100 liquid chromatograph equipped | |
|---|---|---|
| | with solvent delivery system, refrigerated | |
| | autosampler at 5° C., column thermostat, | |
| | degasser and fluorescence detector or equivalent | |
| Column | YMC-PACK ODS-AQ, 250 × 4.6 mm, 5 μm | |
| | particle size (YMC, cod. AQ12S05-2546WT) | |
| Temperature: | 40° C. | |
| Mobile phase: | A: CH₃COONH₄ (1.5 g/L), EDTA (0.55 g/L) | |
| | B: Methanol | |
| Flow rate: | 1 mL/min | |
| Detection (FLD): | wavelength excitation = 275 nm | |
| | wavelength emission = 314 nm | |
| Run Time: | 25 min | |
| Acquisition time: | 6 min | |
| Injection volume: | 20 μL | |
| Reference peak: | Gd(EDTA) | |
| Elution: Gradient | Time (min) | % B |
| | 0 | 0 |
| | 5 | 0 |
| | 10 | 50 |
| | 15 | 50 |
| | 16 | 0 |
| | 25 | 0 |

Solution Preparation

Mobile Phase

In a 1000-mL volumetric flask accurately weigh 1.5 g of ammonium acetate and dissolve with purified water, add 0.70 g of ethylenediaminetetraacetic acid disodium salt dehydrate and then dilute to volume with purified water.

Dilution Solution

In a 1000-mL volumetric flask accurately weigh 3 g of ammonium acetate and dissolve with purified water, add 1.4 g of ethylenediaminetetraacetic acid disodium salt dehydrate and then dilute to volume with purified water.

Blank Solution

Transfer 0.5 mL of purified water in vial, add 0.5 mL of dilution solution. Mix well and inject directly into the chromatographyc system.

Reference Solution

In a 50 mL volumetric flask weight 0.32 g of Gadolinium acetate hydrate (expressed on the anhydrous basis, determine the water content before use) and dilute to volume with mobile phase. The concentration of Gadolinium is 3 mg/mL.

Transfer 0.1 mL of this solution in a 100 mL volumetric flask and dilute to volume with mobile phase. The concentration of Gadolinium is 0.003 mg/mL.

LOQ Solution

Transfer 1 mL of reference solution in a 5 mL volumetric flask and dilute to volume with mobile phase. The concentration of Gadolinium is 0.0006 mg/mL.

Test Solution

In a 10-mL volumetric flask accurately weight 600 mg of the sample under test (expressed on the anhydrous basis) and dilute to volume with purified water. The concentration of dimeric gadolinium complex of Formula I, e.g. of Compound 1, is about 60 mg/mL.

Transfer 0.5 mL of this solution in vial and add 0.5 mL of dilution solution. Mix the sample well. Once diluted immediately place the sample in the refrigerated autosampler (5-8° C.) and inject sample within 5 minutes from dilution. The final concentration of dimeric gadolinium complex of Formula I, e.g. of Compound 1, is about 30 mg/mL.

Analytical Sequence

| | |
|---|---|
| Blank | n = 1 |
| LOQ solution | n = 1 |
| Reference solution | n = 6 |
| Test solutions | n = 6 |
| Reference solution | n = 1 |

System Suitability Test

Carry out the System Suitability Test (SST) every time the method is applied.
After equilibrating the chromatographic system, make one injection of the blank solution and verify the absence of interfering peaks.
Make one injection of LOQ solution

The results of the analytical sequence are valuable if the Gd(EDTA) peak has S/N≥10.
Make six injections of the first reference solution and verify if the following requirements for the Gd(EDTA) peak are satisfied:

| | |
|---|---|
| area repeatability of Gd(EDTA), expressed as percentage | ≤10% |
| relative standard deviation (RSD %, n = 6) | |
| retention time repeatability of Gd(EDTA) peak, expressed as | ≤2% |
| percentage relative standard deviation (RSD %, n = 6) | |
| symmetry factor, T, for Gd(EDTA) peak, | 0.7-2.0 |
| calculated according to Eq. 1 | |

$$T = {w}_{0.05} / 2 f \qquad Eq . 1$$

where:

W_{0.05}=width at one-twentieth of the peak height (min)

f=distance (min) between the perpendicular dropped from the peak maximum and the leading edge of the peak at one-twentieth of the peak height.

Calculation

Calculate the percentage content, Free Gd %, according to Eq.2:

$$Free Gd \% = \frac{{A}_{t} \times {W}_{R S} \times ( 100 - K ) \times 20}{{A}_{std} \times w \times ( 100 - {K}_{1} ) \times 50 \times 1000 \times 2.126} \times 100 \qquad Eq . 2$$

Aₜ=peak area of free Gd in the Test solution
A_{std}=Gd(EDTA) mean area in SST reference solution injections (n=6)
W_{RS}=mg of Gd in the reference solution
w=weight of Compound 1 sample (mg) to prepare the Test solution
K=% of H₂O content in the Gadolinium acetate hydrate
K₁=% of H₂O content in the sample
2.126=correction factor between Gadolinium acetate and Gadolinium

The Limit of Quantitation for free Gd is 0.002% (w/w).

Values lower than the LOQ limit should be expressed as <LOQ or n.q. (not quantifiable).

The percentage calculated as above can be converted in ppm of free-gadolinium vs. complex of Formula I, e.g. vs. Compound 1, by multiplying such percentage * 10,000.

HPLC Procedure 2—Determination of the amount of mono-gadolinated complexes (mono-Gd)

The content of mono-Gd impurities in the dimeric complex of Formula I, e.g. of Compound 1, is quantified by reverse phase HPLC method in the same chromatographic run by using either FLD detector.

Quantification of specified impurity Mono-Gd (in particular, the Mono-Gd complex of Compound 1a with only one gadolinium metal ions) is done by using reference sample Mono-Gd as sodium salt by FLD detection. Mono-Gd sodium salt (reference sample) can be obtained by complexing the dimeric ligand Compound 1a with a less then stoichiometric amount of gadolinium ions to obtain Mono-Gd, adjusting to neutral pH with NaOH and then isolating by concentration to residue.

Chromatographic Conditions

| Instrument: | HPLC Agilent 1100 equipped with solvent | |
|---|---|---|
| | delivery system, autosampler, column thermostat, | |
| | degasser, UV diode array detector and | |
| | fluorescence detector 2475 Waters or equivalent | |
| Column: | Xselect ® HSS T3, 3.5 μm, 150 × 3.0 mm | |
| | (Waters, Part No. 186004781) | |
| Temperature: | 40° C. | |
| Mobile phase: | Solvent A: Mobile phase A (40 mM Potassium | |
| | Phosphate − 0.02 mM EDTA in water, pH 6.2) | |
| | Solvent B: Mobile phase B (Solvent A/ | |
| | Acetonitrile, 60/40 v/v) | |
| Flow rate: | 0.35 mL/min | |
| Detection (FLD): | wavelength excitation (λex) = 275 nm | |
| | wavelength emission (λem) = 314 nm | |
| Detection (UV): | wavelength = 210 nm/Bw: 8 nm; Ref. | |
| | wavelength = 480 nm/Bw: 80 nm | |
| Run Time analysis: | 50 min | |
| Acquisition time: | 32 min | |
| Injection volume: | 10 μL | |
| Eluition: Gradient | Time (min) | % B |
| | 0 | 0 |
| | 4 | 0 |
| | 20 | 15 |
| | 25 | 15 |
| | 30 | 100 |
| | 40 | 100 |
| | 43 | 0 |
| | 50 | 0 |

Solution Preparation

Mobile Phase A

In a 2000-mL volumetric flask accurately weigh:
8.56 g of Potassium di-hydrogen phosphate
3.97 g of Di-Potassium hydrogen phosphate tri-hydrate
0.015 g of Titriplex® III (EDTA disodium salt) and then dilute to volume with purified water. Filter through a 0.22 μm membrane filter.

Mobile Phase B

In a 1000-mL volumetric flask transfer 600 mL of Mobile Phase A and dilute to volume with Acetonitrile. Mix well.

Solution of CaCl₂

In a 50-mL volumetric flask accurately weigh 165 mg of CaCl₂ (expressed on the anhydrous basis) and dilute to volume with purified water.

The concentration is about 3.3 mg/mL.

Stock solution of Mono-Gd

In a 50-mL volumetric flask accurately weight 25 mg of Mono-Gd sodium salt (expressed on the anhydrous basis and purity) and dilute to volume with purified water.

The concentration of Mono-Gd is about 0.5 mg/mL

Weight of Mono-Gd=Weight of Mono-Gd sodium salt * 1140.31/1162.29

Reference Solution of Mono-Gd

In a 5-mL volumetric flask accurately transfer 0.45 mL of the stock solution of Mono-Gd. Add 1 mL of CaCl₂ solution and dilute to volume with purified water. The concentration of the standard is 0.045 mg/mL.

LoQ solution of Mono-Gd

In a 5-mL volumetric flask accurately transfer 0.1 mL of the stock solution of Mono-Gd. Add 1 mL of CaCl₂ solution and dilute to volume with purified water. The concentration of Mono-Gd is 0.01 mg/mL.

Blank Solution

Transfer 0.8 mL of water solution in vial, add 0.2 mL of CaCl₂ solution. Mix well.

Test Solution

In a 5-mL volumetric flask accurately weight 125 mg of the sample under test (expressed on the anhydrous basis). Add 1 mL of CaCl₂ solution and dilute to volume with purified water. The concentration of Compound 1 is about 25 mg/mL.

Analytical Sequence

| | |
|---|---|
| Blank | n = 1 |
| LoQ solution | n = 1 |
| Reference solution | n = 6 |
| Test solutions | n = 6 |
| Reference solution | n = 1 |

Calculation

Calculate the percentage content of Mono-Gd by FLD acquisition, according o Eq. 3

$${C}_{MonoGd} \% \frac{w}{w} = \frac{{A}_{T} \times {V}_{S} \times {W}_{S} \times ( 100 - K F ) \times a \times f}{{A}_{R} \times {w}_{T} \times 50 \times 100 \times ( 100 - K {F}_{{Compound}_{1}} )} \times 100 \qquad Eq . 3$$

where:
A_{T}: Peak area (area sum of Mono-Gd-1/2/3/4 peaks, if present) in the Test solution
A_{R}: Peak area (area sum of Mono-Gd-1/2/3/4 peaks) in the Reference solution injections (mean value n=6)
W_{T}: Weight of the sample in the Test solution (mg)
Vₛ: Volume of the Mono-Gd stock solution withdrawn to prepare the Reference solution (mL)
Wₛ: Weight of the Mono-Gd sodium salt used to prepare the stock solution (mg)
KF: % of H₂O content in Mono-Gd sodium salt
KF_{Compound1}:/% of H₂O content in Compound 1
a: % assay of Mono-Gd sodium salt
f: correction factor for molecular weight: 0.98

The Limit of Quantitation for Mono-Gd (sum of four peaks) is 0.04% (w/w).

Values lower than the LOQ limit should be expressed as <LOQ or n.q. (not quantifiable).

The percentage calculated as above can be converted in ppm of mono-gadolinated complex vs. complex of Formula I, e.g. vs. Compound 1, by multiplying such percentage * 10,000.

1. A process for the manufacturing of a solution of the gadolinium complex of formula I 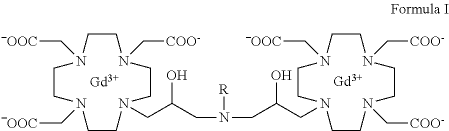
wherein R is a C₃-C₁₂ hydroxyalkyl comprising at least 2 hydroxyl groups;
comprising the following steps:
i) providing a solution of a dimeric ligand of formula Ia 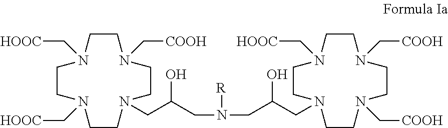
wherein R is as defined above,
ii) adding to the solution of the previous step a molar excess of gadolinium metal ions to complex the dimeric ligand provided in step i), whereby an intermediate solution comprising the gadolinium complex of formula I is obtained, and
iii) adding to the intermediate solution of the previous step at least a precipitating agent selected from the group consisting of phosphate (PO₄³⁻), monohydrogen phosphate (1PO₄²⁻), dihydrogen phosphate (H₂PO₄₋), orthophosphoric acid (H₃PO₄), oxalate (C₂O₄²⁻), hydrogen oxalate (HC₂0₄₋), and oxalic acid (H₂C₂O₄) to precipitate a portion of free gadolinium metal ions as gadolinium salt, whereby a portion of free gadolinium metal ions precipitate as gadolinium salt and said solution of gadolinium complex of formula I is obtained,
wherein the precipitating agent is added in an amount from 1.1 to 5 moles with respect to 1 mole of gadolinium metal ions within the intermediate solution.

2. The process according to claim 1, wherein R of formulae I and Ia is a C₅-C₇ polyol.

3. The process according to claim 2, wherein the gadolinium complex is the complex Compound 1 having the following formula 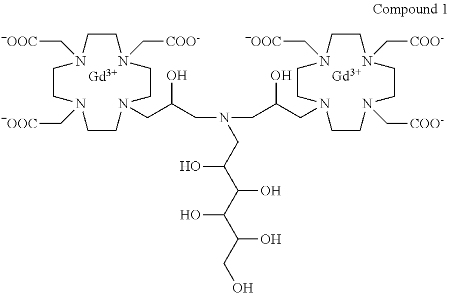
and the dimeric ligand is the ligand Compound 1a having the following formula:

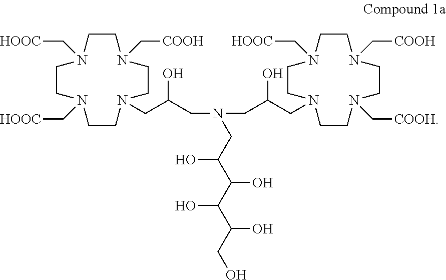 [SMILES: O=C(O)CN1CCN(CC(=O)O)CCN(CC(O)CN(CC(O)CN2CCN(CC(=O)O)CCN(CC(=O)O)CCN(CC(=O)O)CC2)CC(O)C(O)C(O)C(O)CO)CCN(CC(=O)O)CC1]

4. The process according to claim 1 wherein the precipitating agent is selected from the group consisting of phosphate (PO₄³⁻), oxalate (C₂O₄²⁻) and monohydrogen phosphate (HPO₄²⁻)

5. The process according to claim 1,
wherein the precipitating agent is added by adding to the intermediate solution a precipitating salt comprising the precipitating agent.

6. The process according to claim 1, wherein the precipitating agent is added in an amount from 1.2 to 3 moles, with respect to 1 mole of gadolinium metal ions within the intermediate solution.

7. The process according to claim 6, wherein the precipitating agent is added in an amount from 1.4 to 2.5 moles, with respect to 1 mole of gadolinium metal ions within the intermediate solution.

8. The process according to claim 7, wherein the precipitating agent is added in an amount from 1.4 to 1.6 moles, with respect to 1 mole of gadolinium metal ions within the intermediate solution.

9. The process according to claim 1, wherein during and/or after the addition of the precipitating agent according to step iii), the pH is adjusted to and/or maintained at a value of 4.5 or higher.

10. The process according to claim 9, wherein during and/or after the addition of the precipitating agent according to step iii), the pH is adjusted to and/or maintained at a value of 4.7 or higher.

11. The process according to claim 10, wherein during and/or after the addition of the precipitating agent according to step iii), the pH is adjusted to and/or maintained at a value of 4.9 or higher.

12. The process according to claim 11, wherein during and/or after the addition of the precipitating agent according to step iii), the pH is adjusted to and/or maintained at a value of 5.5 or higher.

13. The process according to claim 1, wherein during and/or after the addition of the precipitating agent according to step iii), the pH is adjusted to and/or maintained at a value of 10.0 or lower.

14. The process according to claim 13, wherein during and/or after the addition of the precipitating agent according to step iii), the pH is adjusted to and/or maintained at a value of 9.0 or lower.

15. The process according to claim 14, wherein during and/or after the addition of the precipitating agent according to step iii), the pH is adjusted to and/or maintained at a value of 8.5 or lower.

16. The process according to claim 15, wherein during and/or after the addition of the precipitating agent according to step iii), the pH is adjusted to and/or maintained at a value of 7.5 or lower.

17. The process according to claim 16, wherein during and/or after the addition of the precipitating agent according to step iii), the pH is adjusted to and/or maintained at a value of 6.5 or lower.

18. The process according to claim 1, wherein the solution of the dimeric ligand provided in step i) is obtained according to the following steps:
a) providing a solution of the protected dimeric ligand of formula Ib 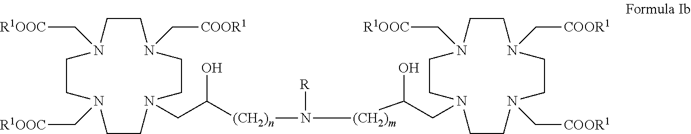
wherein R is a C₃-C₁₂ hydroxyalkyl comprising at least 2 hydroxyl groups;
n and m are 1; and
R¹ is a C₁-C₆ alkyl,
b) adding an acid to the solution of the previous step to lower the pH thereof;
c) during and/or after step b), heating and/or maintaining the temperature of the reaction mixture to a temperature higher than 40° C. to deprotect the protected dimeric ligand, whereby a solution comprising the dimeric ligand is obtained; and
d) optionally adding a base to the solution comprising the dimeric ligand obtained in step c) to protonate the carboxyl groups of the dimeric ligand.

19. The process according to claim 1, further comprising at least one further purification step after step iii) to further reduce the residual amount of free gadolinium metal ions after step iii).

20. The process according to claim 18 wherein, after carrying out step iii) and before carrying out the optional at least one further purification step, the amount of free gadolinium metal ions within such solution of gadolinium complex is less than 350 ppm vs. the amount of gadolinium complex, and/or the amount of mono-gadolinated complex within such solution of gadolinium complex is lower than 550 ppm vs. the amount of gadolinium complex.

21. A solution of gadolinium complex of formula I 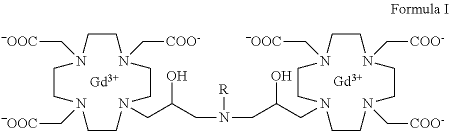
wherein R is a C₃-C₁₂ hydroxyalkyl comprising at least 2 hydroxyl groups; comprising an amount of free gadolinium metal ions less than 350 ppm vs. the amount of gadolinium complex, and an amount of mono-gadolinated complex less than 550 ppm vs. the amount of gadolinium complex.

22. A solution of gadolinium complex of formula I 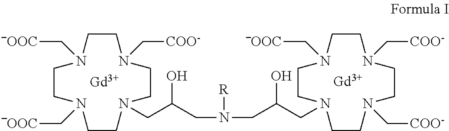
wherein R is a C₃-C₁₂ hydroxyalkyl comprising at least 2 hydroxyl groups; comprising an amount of free gadolinium metal ions less than 350 ppm, obtainable according to the process of claim 1.

23. A process for the manufacturing of an isolated gadolinium complex of formula I 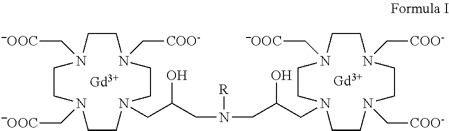
wherein R is a C₃-C₁₂ hydroxyalkyl comprising at least 2 hydroxyl groups,
comprising the process of manufacturing of a solution of the gadolinium complex according to claim 1, and further step iv):
iv) isolating the gadolinium complex from said solution of the gadolinium complex.

24. An isolated gadolinium complex of formula I 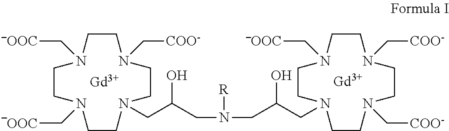
wherein R is a C₃-C₁₂ hydroxyalkyl comprising at least 2 hydroxyl groups, obtainable according to the process of claim 23.

25. The process according to claim 1, wherein R of formulae I and Ia is selected from a pentyl-tetraol of formula 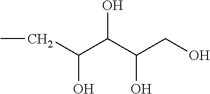
and a hexyl-pentaol of formula

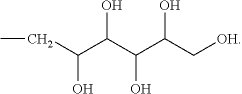

26. The process according to claim 5, wherein the precipitating salt is selected from the group consisting of sodium phosphate (Na₃PO₄), potassium phosphate (K₃PO₄), sodium hydrogen phosphate (Na₂HPO₄), potassium hydrogen phosphate (K₂HIPO4), sodium dihydrogen phosphate (NaH₂PO₄), potassium dihydrogen phosphate (KH₂PO₄), sodium oxalate (Na₂C₂O₄), potassium oxalate (K₂C₂O₄), sodium hydrogen oxalate (NaHC₂O₄), and potassium hydrogen oxalate (KHC₂O₄₋).

27. The process according to claim 19, wherein said further purification step comprises loading the solution of gadolinium complex onto an adsorbent resin whereby a further portion of residual free gadolinium metal ions is removed from the solution.
